(19) United States
(12) Patent Application Publication
(10) Pub. No.: US 2004/0157281 A1
(43) Pub. Date: Aug. 12, 2004

(54) Method and apparatus for detecting an analyte

(75) Inventors: Keren I. Hulkower, Evanston, IL (US); Avijit Sen, Champaign, IL (US); Joel S. Dryer, Northbrook, IL (US)

Correspondence Address: BANNER & WITCOFF, LTD., TEN SOUTH WACKER DRIVE, SUITE 3000, CHICAGO, IL, 60606, US

(73) Assignee: ChemSensing, Inc., Northbrook, IL

(21) Appl. No.: 10/775,780

(22) Filed: Feb. 10, 2004

Related U.S. Application Data
(60) Provisional application No. 60/446,585, filed on Feb. 11, 2003.
(60) Provisional application No. 60/447,161, filed on Feb. 13, 2003.

(51) Int. Cl. C12Q 1/68; G01N 33/53; G01N 33/537; G01N 33/543

(52) U.S. Cl. 435/7.92; 435/6

(57) ABSTRACT

A detection protocol or process and apparatus or device for practicing such a process for detecting an analyte comprising an analyte-specific compound that binds to the analyte, and producing a detectable compound, the detectable compound producing a response when exposed to at least one porphyrin dye or at least two dyes, the response being stronger and more distinct than a response of the analyte when exposed to the at least one porphyrin dye or the at least two dyes.

### CONTINUING APPLICATION DATA

This application claims priority to provisional application Serial No. 60/446,585, filed Feb. 11, 2003, and provisional application Serial No. 60/447,161, filed Feb. 13, 2003, both of which are incorporated by reference in their entireties.

### FIELD OF THE INVENTION

The present invention relates to methods and apparatus for analyte or compound detection by means of chemical processes that produce a visual display response to the presence of the analyte or compound.

### BACKGROUND OF THE INVENTION

There are a number of conventional methods and apparatuses used to detect an analyte or compound. See e.g. the Background Section of U.S. Pat. Nos. 6,368,558 and 6,495,102. Detection of analyte or compounds can be limited, and in some cases not possible, using conventional methods and apparatuses due to the nature of analytes or compounds. Thus, there is a need for methods and apparatuses for detection that take into account the nature of the analyte or compound. There is a further need for methods and apparatuses that take into account the nature of the analyte or compound and that expand and/or improve the uses and applications of existing methods and apparatuses, including but not limited to, artificial olfaction methods and artificial noses and artificial tongues.

### BRIEF SUMMARY OF THE INVENTION

The present invention comprises a detection protocol or process for detecting and identifying an analyte as well as apparatus or devices for practicing such a process. In one embodiment, the present invention provides a method and device for detecting an analyte comprising an analyte-specific compound that binds to the analyte and produces a detectable compound, said detectable compound producing a response when exposed to at least one porphyrin dye or at least two dyes, the response being stronger and more distinct than a response of the analyte when exposed to the at least one porphyrin dye or the at least two dyes.

In one embodiment, the present invention provides an analyte-specific compound that binds to the analyte and produces a detectable compound in combination with a given substrate. The detectable compound, when exposed to at least one porphyrin dye or at least two dyes, produces a response by the dye(s) thereby indicating analyte presence qualitatively, and in some circumstances quantitatively. The response of the dye(s) to the detectable compound is stronger and more distinct than a response of the analyte when exposed to the dye(s). In one embodiment, an enzyme is chemically coupled to the analyte-specific compound.

In one embodiment, the present invention provides a device for detecting an analyte comprising an analyte-specific compound conjugated to an enzyme, the analyte-specific compound binding to a target site of the analyte, the enzyme producing a detectable compound in combination with a given substrate, the detectable compound producing a detectable response when exposed to at least one porphyrin dye or at least two dyes, the response being stronger and more distinct than a response of the analyte when exposed to the at least one porphyrin dye or the at least two dyes. The device for this embodiment can further comprise a capture analyte-specific compound that is different from the conjugated analyte-specific compound, the capture analyte-specific compound binding to a different target site of the analyte than the conjugated analyte-specific compound.

In one embodiment, the present invention provides a device for detecting an analyte comprising an analyte-specific compound that binds to a target site of the analyte, a conjugate comprising an enzyme and a non-analyte specific compound, the non-analyte specific compound that binds to the analyte-specific compound, the enzyme producing a detectable compound in combination with a given substrate, the detectable compound producing a detectable response when exposed to at least one porphyrin dye or the at least two dyes, the response being stronger and more distinct than a response of the analyte when exposed to the at least one porphyrin dye or the at least two dyes. The device for this embodiment can further comprise a capture analyte-specific compound that is different from the analyte-specific compound, the capture analyte-specific compound binding to a different target site of the analyte than the analyte-specific compound. One skilled in the art will recognize that this embodiment can be incorporated into a wide variety of enzyme immunoassay formats, including but not limited to enzyme-linked immunosorbent assays (ELISA), or competitive enzyme immunoassays, or lateral flow immunoassays.

In one embodiment, the present invention provides a method for detecting an analyte comprising the steps of: a) exposing an analyte-specific compound to an analyte; b) producing a detectable compound; and c) exposing the detectable compound to at least one porphyrin dye or at least two dyes to produce a response, the response being stronger and more distinct than a response of the analyte when exposed to the at least one porphyrin dye or the at least two dyes.

In one embodiment, the present invention provides a method for detecting an analyte comprising the steps of: a) exposing an analyte-specific compound to an analyte; and b) producing a detectable compound, in combination with a given substrate, said detectable compound producing a response when exposed to at least one porphyrin dye or at least two dyes, the response being stronger and more distinct than a response of the analyte when exposed to the at least one porphyrin dye or the at least two dyes.

In one embodiment, the present invention provides a method for detecting an analyte comprising the steps of: a) exposing the analyte to a conjugate, the conjugate comprising an analyte-specific compound conjugated to an enzyme; b) exposing the conjugate to a given substrate to produce a detectable compound, the detectable compound producing a detectable response when exposed to at least one porphyrin dye or at least two dyes, the response being stronger and more distinct than a response of the analyte when exposed to the at least one porphyrin dye or the at least two dyes. This method can further comprise the step of removing unbound material prior to step b).

In one embodiment, the present invention provides a method for detecting an analyte comprising the steps of: a) exposing the analyte to an analyte-specific compound; b) exposing the analyte-specific compound to a conjugate comprising an enzyme and a non-analyte specific compound, the non-analyte specific compound binding to the analyte-specific compound; and c) exposing the enzyme to a given substrate to produce a detectable compound, the detectable compound producing a detectable response when exposed to at least one porphyrin dye or at least two dyes, the response being stronger and more distinct than a response of the analyte when exposed to the at least one porphyrin dye or the at least two dyes. This method can further comprise the step of removing unbound material prior to steps b) and c).

In another embodiment, the present invention comprises a competitive binding process or device for detecting an analyte in a sample comprising a receptor molecule for capturing either the free analyte from a sample, or a tracer not from the sample, the tracer comprising an analyte molecule bound to an enzyme, the tracer capable of producing a detectable compound in combination with a given substrate, the detectable compound producing a detectable response when exposed to at least one porphyrin dye or at least two dyes, the detectable response inversely proportional to the quantity of the analyte in the sample.

Examples of this invention include, but are not limited to, an immunoassay or nucleic acid detection assay, the immunoassay or nucleic acid detection assay having an enzyme component, the enzyme capable of catalyzing a specific chemical reaction when exposed to a specific substrate, the chemical reaction producing a detectable compound corresponding (i.e. proportional or inversely proportional) to the analyte. Another example of this invention includes the detection of target nucleic acid sequences as analytes using DNA, RNA, or chimeric sequence specific probes conjugated to enzymes.

The detectable compound produced in accordance with the present invention can be detected using any suitable method and artificial nose or tongue, including but not limited to those methods and devices disclosed in U.S. Pat. Nos. 6,368,558 and 6,495,102, and U.S. Patent Application Publication Nos. US 2003/0129085 A1, US 2003/0143112 A1, and US 2003/0166298 A1, all of which are incorporated herein by reference. For example, one or more porphyrin dyes in its metalated form (e.g., metalloporphyrin) or non-metalated form (e.g., free-base porphyrin) can be used to detect the enzymatically generated volatile compounds and the porphyrin dye can be used alone or as a component in an array with other chemical dyes, such as Bronsted acid-base dyes, Lewis acid-base dyes, zwitterionic solvatochromic dyes, and other chemoresponsive dyes. Those skilled in the art will recognize that any suitable method may be used to detect and quantitate a dye color change corresponding to the presence of the enzymatically generated volatile compound, such as the unassisted eye, spectrophotometry and fluorescence detection or other readers or sensors.

The enzyme used in accordance with the examples of the present invention can be either a free enzyme or a conjugated enzyme (sometimes called an enzyme conjugate).

Conjugated enzymes are conjugated to any suitable molecule, such as an antibody, protein, biotin, peptide, hapten, specific drug analyte or drug metabolite, carbohydrate moiety, or single stranded or double stranded nucleic acids of any given length or base sequence, including but not limited to RNA, DNA, base-modified or chimeric oligonucleotides, amplification reaction products and cDNA. Conjugated enzymes can also be conjugated to fusion or chimeric molecules comprising structural elements of two or more classes of molecules such as combinations of nucleic acid and protein or protein and small organic molecules or the like.

In accordance with the present invention, the free enzyme or the enzyme conjugate and its cognate substrate are placed on a suitable solid support or in a suitable aqueous or organic solution. In accordance with the present invention, a suitable solid support is a membrane, filter, tube, well, or plate. Examples of a material for a suitable solid support include polystyrene and/or polypropylene. An example of a suitable aqueous or organic solution is a buffer solution.

The present invention provides methods and devices for analyte detection that take into account the nature of the analyte. Those skilled in the art will recognize the many and varied uses and applications for the present invention, as well as the advantages of the present invention not attained previously by existing methods and devices.

### BRIEF DESCRIPTION OF THE DRAWINGS

FIG. 1 illustrates the manufacture and detection of volatile compounds produced by enzyme catalyzed reactions in accordance with the present invention.

FIG. 2a illustrates an analyte detection assay in accordance with the present invention.

FIG. 2b illustrates another analyte detection assay in accordance with the present invention.

FIG. 3a illustrates another analyte detection assay in accordance with the present invention.

FIG. 3b illustrates another analyte detection assay in accordance with the present invention.

FIG. 4 illustrates a competitive binding test for an analyte in accordance with the present invention.

FIG. 5 illustrates a lateral flow assay in accordance with the present invention.

FIG. 6 illustrates an array-based quantification of volatile compounds made in accordance with the present invention used to detect analytes in a well of an enzyme immunoassay in a multi-well plate, in a competitive binding assay for an analyte, and in a lateral flow assay, all in accordance with the present invention.

FIG. 7 illustrates the detection of a protein on an Immunoblot membrane in accordance with the present invention.

### DETAILED DESCRIPTION OF THE INVENTION

The present invention comprises a detection protocol or process for detecting and identifying an analyte as well as apparatus or devices for practicing such a process. In one embodiment, the present invention provides a method and device for detecting an analyte comprising an analyte-specific compound that binds to the analyte and produces a detectable compound, said detectable compound producing a response when exposed to at least one porphyrin dye or at least two dyes, the response being stronger and more distinct than a response of the analyte when exposed to the at least one porphyrin dye or the at least two dyes. In the embodiment relating to the at least one porphyrin dye, the at least one porphyrin dye may be a porphyrin dye in its metalated form (e.g., metalloporphyrin) or non-metalated form (e.g., free-base porphyrin). In the embodiment relating to at least two dyes, the at least two dyes may be any suitable dyes that provide produce a response when exposed to the detectable compound, including porphyrin dyes and/or non-porphyrin dyes, or any chemoresponsive dyes.

In one embodiment, the present invention provides an analyte-specific compound that binds to the analyte and produces a detectable compound in combination with a given substrate. The detectable compound, when exposed to at least one porphyrin dye or at least two dyes, produces a response by the dye(s) thereby indicating analyte presence qualitatively, and in some circumstances quantitatively. The response of the dye(s) to the detectable compound is stronger and more distinct than a response of the analyte when exposed to the dye(s). In one embodiment, an enzyme is chemically coupled to the analyte-specific compound. In the embodiment relating to the at least one porphyrin dye, the at least one porphyrin dye may be a porphyrin dye in its metalated form (e.g., metalloporphyrin) or non-metalated form (e.g., free-base porphyrin). In the embodiment relating to at least two dyes, the at least two dyes may be any suitable dyes that provide produce a response when exposed to the detectable compound, including porphyrin dyes and/or non-porphyrin dyes, or any chemoresponsive dyes.

In one embodiment, the present invention provides a device for detecting an analyte comprising an analyte-specific compound conjugated to an enzyme, the analyte-specific compound binding to a target site of the analyte, the enzyme producing a detectable compound in combination with a given substrate, the detectable compound producing a detectable response when exposed to at least one porphyrin dye or at least two dyes, the response being stronger and more distinct than a response of the analyte when exposed to the at least one porphyrin dye or the at least two dyes. The device for this embodiment can further comprise a capture analyte-specific compound that is different from the conjugated analyte-specific compound, the capture analyte-specific compound binding to a different target site of the analyte than the conjugated analyte-specific compound. In the embodiment relating to the at least one porphyrin dye, the at least one porphyrin dye may be a porphyrin dye in its metalated form (e.g., metalloporphyrin) or non-metalated form (e.g., free-base porphyrin). In the embodiment relating to at least two dyes, the at least two dyes may be any suitable dyes that provide produce a response when exposed to the detectable compound, including porphyrin dyes and/or non-porphyrin dyes, or any chemoresponsive dyes.

In one embodiment, the present invention provides a device for detecting an analyte comprising an analyte-specific compound that binds to a target site of the analyte, a conjugate comprising an enzyme and a non-analyte specific compound, the non-analyte specific compound that binds to the analyte-specific compound, the enzyme producing a detectable compound in combination with a given substrate, the detectable compound producing a detectable response when exposed to at least one porphyrin dye or at least two dyes, the response being stronger and more distinct than a response of the analyte when exposed to the at least one porphyrin dye or the at least two dyes. The device for this embodiment can further comprise a capture analyte-specific compound that is different from the analyte-specific compound, the capture analyte-specific compound binding to a different target site of the analyte than the analyte-specific compound. One skilled in the art will recognize that this embodiment can be incorporated into a wide variety of enzyme immunoassay formats, including but not limited to enzyme-linked immunosorbent assays (ELISA), or competitive enzyme immunoassays, or lateral flow immunoassays. In the embodiment relating to the at least one porphyrin dye, the at least one porphyrin dye may be a porphyrin dye in its metalated form (e.g., metalloporphyrin) or non-metalated form (e.g., free-base porphyrin). In the embodiment relating to at least two dyes, the at least two dyes may be any suitable dyes that provide produce a response when exposed to the detectable compound, including porphyrin dyes and/or non-porphyrin dyes, or any chemoresponsive dyes.

In one embodiment, the present invention provides a method for detecting an analyte comprising the steps of: a) exposing an analyte-specific compound to an analyte; b) producing a detectable compound; and c) exposing the detectable compound to at least one porphyrin dye or at least two dyes to produce a response, the response being stronger and more distinct than a response of the analyte when exposed to the at least one porphyrin dye or the at least two dyes. In the embodiment relating to the at least one porphyrin dye, the at least one porphyrin dye may be a porphyrin dye in its metalated form (e.g., metalloporphyrin) or non-metalated form (e.g., free-base porphyrin). In the embodiment relating to at least two dyes, the at least two dyes may be any suitable dyes that provide produce a response when exposed to the detectable compound, including porphyrin dyes and/or non-porphyrin dyes, or any chemoresponsive dyes.

In one embodiment, the present invention provides a method for detecting an analyte comprising the steps of: a) exposing an analyte-specific compound to an analyte; and b) producing a detectable compound, in combination with a given substrate, said detectable compound producing a response when exposed to at least one porphyrin dye or at least two dyes, the response being stronger and more distinct than a response of the analyte when exposed to the at least one porphyrin dye or the at least two dyes. In the embodiment relating to the at least one porphyrin dye, the at least one porphyrin dye may be a porphyrin dye in its metalated form (e.g., metalloporphyrin) or non-metalated form (e.g., free-base porphyrin). In the embodiment relating to at least two dyes, the at least two dyes may be any suitable dyes that provide produce a response when exposed to the detectable compound, including porphyrin dyes and/or non-porphyrin dyes, or any chemoresponsive dyes.

In one embodiment, the present invention provides a method for detecting an analyte comprising the steps of: a) exposing the analyte to a conjugate, the conjugate comprising an analyte-specific compound conjugated to an enzyme; b) exposing the conjugate to a given substrate to produce a detectable compound, the detectable compound producing a detectable response when exposed to at least one porphyrin dye or at least two dyes, the response being stronger and more distinct than a response of the analyte when exposed to the at least one porphyrin dye or the at least two dyes. This method can further comprise the step of removing unbound material prior to step b). In the embodiment relating to the at least one porphyrin dye, the at least one porphyrin dye may be a porphyrin dye in its metalated form (e.g., metalloporphyrin) or non-metalated form (e.g., free-base porphyrin). In the embodiment relating to at least two dyes, the at least two dyes may be any suitable dyes that provide produce a response when exposed to the detectable compound, including porphyrin dyes and/or non-porphyrin dyes, or any chemoresponsive dyes.

In one embodiment, the present invention provides a method for detecting an analyte comprising the steps of: a) exposing the analyte to an analyte-specific compound; b) exposing the analyte-specific compound to a conjugate comprising an enzyme and a non-analyte specific compound, the non-analyte specific compound binding to the analyte-specific compound; and c) exposing the enzyme to a given substrate to produce a detectable compound, the detectable compound producing a detectable response when exposed to at least one porphyrin dye or at least two dyes, the response being stronger and more distinct than a response of the analyte when exposed to the at least one porphyrin dye or the at least two dyes. This method can further comprise the step of removing unbound material prior to steps b) and c). In the embodiment relating to the at least one porphyrin dye, the at least one porphyrin dye may be a porphyrin dye in its metalated form (e.g., metalloporphyrin) or non-metalated form (e.g., free-base porphyrin). In the embodiment relating to at least two dyes, the at least two dyes may be any suitable dyes that provide produce a response when exposed to the detectable compound, including porphyrin dyes and/or non-porphyrin dyes, or any chemoresponsive dyes.

In another embodiment, the present invention comprises a competitive binding process or device for detecting an analyte in a sample comprising a receptor molecule for capturing either the free analyte from a sample, or a tracer not from the sample, the tracer comprising an analyte molecule bound to an enzyme, the tracer capable of producing a detectable compound in combination with a given substrate, the detectable compound producing a detectable response when exposed to at least one porphyrin dye or at least two dyes, the detectable response inversely proportional to the quantity of the analyte in the sample. In the embodiment relating to the at least one porphyrin dye, the at least one porphyrin dye may be a porphyrin dye in its metalated form (e.g., metalloporphyrin) or non-metalated form (e.g., free-base porphyrin). In the embodiment relating to at least two dyes, the at least two dyes may be any suitable dyes that provide produce a response when exposed to the detectable compound, including porphyrin dyes and/or non-porphyrin dyes, or any chemoresponsive dyes.

Examples of this invention include, but are not limited to, an immunoassay or nucleic acid detection assay, the immunoassay or nucleic acid detection assay having an enzyme component, the enzyme capable of catalyzing a specific chemical reaction when exposed to a specific substrate, the chemical reaction producing a detectable compound corresponding (i.e. proportional or inversely proportional) to the analyte. Another example of this invention includes the detection of target nucleic acid sequences as analytes using DNA, RNA, or chimeric sequence specific probes conjugated to enzymes.

The detectable compound produced in accordance with the present invention can be detected using any suitable method and artificial nose or tongue, including but not limited to those methods and devices disclosed in U.S. Pat. Nos. 6,368,558 and 6,495,102, and U.S. Patent Application Publication Nos. US 2003/0129085 A1, US 2003/0143112 A1, and US 2003/0166298 A1, all of which are incorporated herein by reference. For example, one or more porphyrin dyes in its metalated form (e.g., metalloporphyrin) or non-metalated form (e.g., free-base porphyrin) can be used to detect the enzymatically generated volatile compounds and the porphyrin dye can be used alone or as a component in an array with other chemical dyes, such as Bronsted acid-base dyes, Lewis acid-base dyes, zwitterionic solvatochromic dyes, and other chemoresponsive dyes. Those skilled in the art will recognize that any suitable method may be used to detect and quantitate a dye color change corresponding to the presence of the enzymatically generated volatile compound, such as the unassisted eye, spectrophotometry and fluorescence detection or other readers or sensors.

The enzyme used in accordance with the examples of the present invention can be either a free enzyme or a conjugated enzyme (sometimes called an enzyme conjugate). Conjugated enzymes are conjugated to any suitable molecule, such as an antibody, protein, biotin, peptide, hapten, specific drug analyte or drug metabolite, carbohydrate moiety, or single stranded or double stranded nucleic acids of any given length or base sequence, including but not limited to RNA, DNA, base-modified or chimeric oligonucleotides, amplification reaction products and cDNA. Conjugated enzymes can also be conjugated to fusion or chimeric molecules comprising structural elements of two or more classes of molecules such as combinations of nucleic acid and protein or protein and small organic molecules or the like.

In accordance with the present invention, the free enzyme or the enzyme conjugate and its cognate substrate are placed on a suitable solid support or in a suitable aqueous or organic solution. In accordance with the present invention, a suitable solid support is a membrane, filter, tube, well, or plate. Examples of a material for a suitable solid support include polystyrene and/or polypropylene. An example of a suitable aqueous or organic solution is a buffer solution.

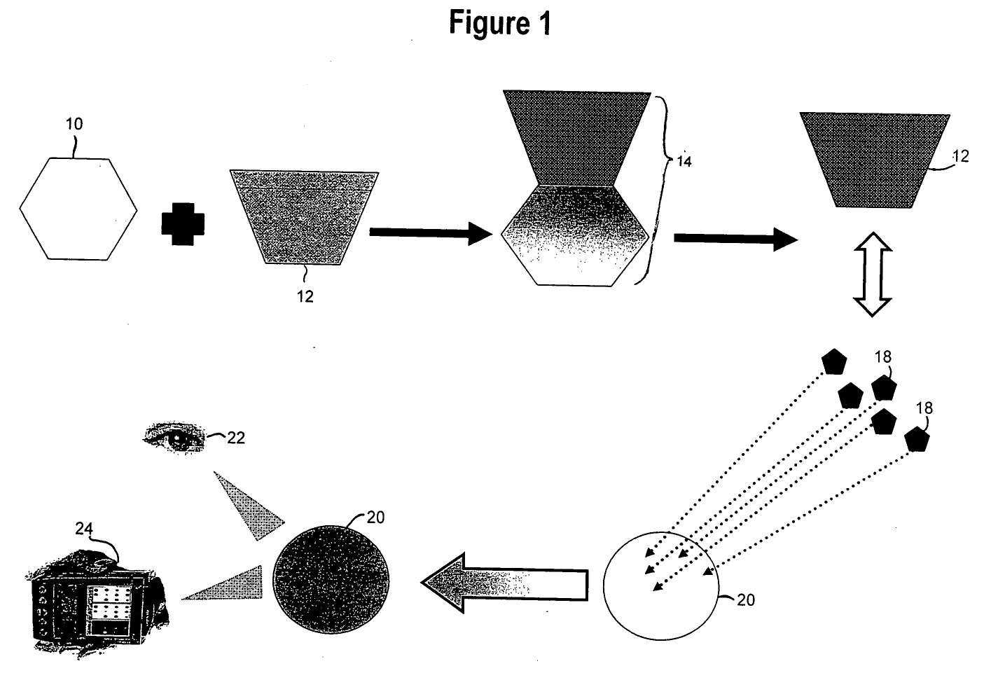

FIG. 1 illustrates the manufacture and detection of volatile compounds produced by enzyme catalyzed reactions in accordance with the present invention. A cognate substrate 10 is combined with an enzyme 12. The combination 14 of cognate substrate 10 and enzyme 12 produces detectable or volatile products or compounds 18. When dye 20 is then exposed to detectable compounds 18, dye 20 undergoes a color change corresponding to presence of detectable compounds 18. The color change of dye 20 can be detected by the human eye 22 or a detector 24. Dye 20 and/or detector 24 can be any suitable dye or detector, including but not limited to the dyes and devices disclosed in U.S. Pat. Nos. 6,368,558, and 6,495,102, and U.S. Patent Application Publication Nos. US 2003/0129085 A1, US 2003/0143112 A1, and US 2003/0166298 A1, all of which are incorporated herein by reference. Examples of suitable dyes are porphyrin dyes, Bronsted acid-base dyes, Lewis acid dyes, solvatorchromic dyes, and other chemoresponsive dyes.

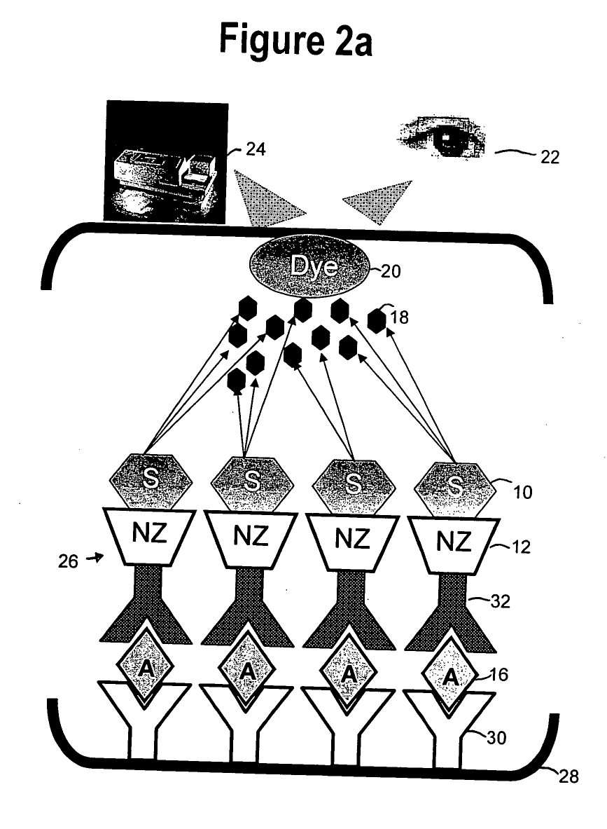

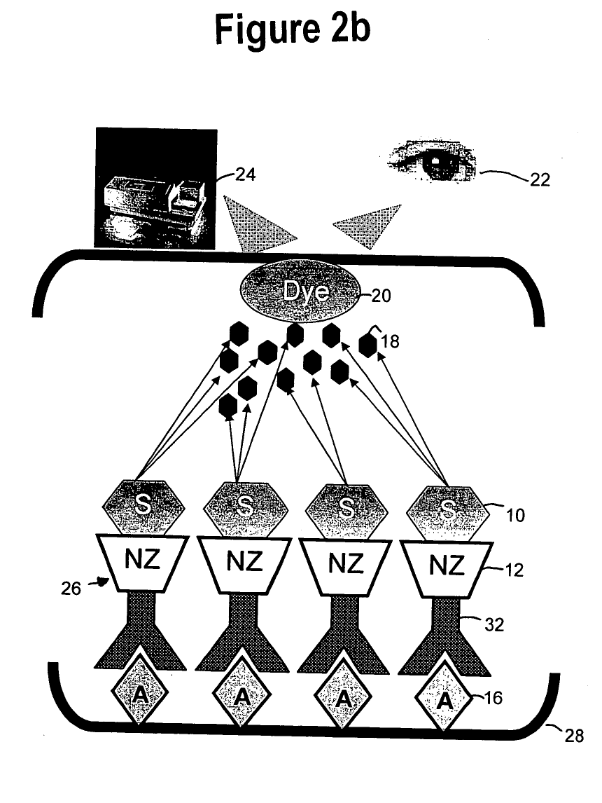

By way of example, the present invention can be used to detect an analyte 16, such as a captured antigen as shown in FIGS. 2 through 7, by using an immunoconjugate, such as an antibody coupled to urease. By way of example, the enzyme 12 can be urease, the cognate substrate 10 can be urea and the detectable compound 18 can be a volatile compound, such as ammonia. The ammonia is produced by an enzyme and substrate combination of urease and urea. If an antigen is present, the antibody-urease complex will bind to it, and the substrate urea, when added, will be enzymatically converted to ammonia. The ammonia is then detected by a color change in a dye 20, such as a porphyrin dye.

An enzyme immunoassay using one antibody for antigen capture and one immunoconjugated antibody for detection is shown in FIG. 2a. An enzyme immunoassay 26 (also referred to as “EIA”) comprises a well 28 that is coated with a capture analyte-specific compound 30. In one embodiment, capture analyte-specific compound 30 is a capture antibody. Capture analyte-specific compound 30 binds to an epitope or target site of analyte 16. An analyte-specific compound 32 is conjugated to enzyme 12. Conjugated analyte-specific compound 32 is different from capture analyte-specific compound 30, and binds to a different and distinct epitope or target site of analyte 16 than the capture analyte-specific compound. In one embodiment, conjugated analyte-specific compound 32 is a specific antibody that is different from the capture antibody.

If no analyte 16 is present in a subject sample, conjugated analyte-specific compound 32 has no analyte 16 to bind to, and the conjugated analyte-specific compound 32 along with the conjugated enzyme 12 is removed from immunoassay 26 using any suitable process (e.g., washing away with a buffer) that would not remove conjugated analyte-specific compound 32 if it had been bound to analyte 16.

If analyte 16 is present in the subject sample, conjugated analyte-specific compound 32 will bind to analyte 16, and enzyme 12 will thus be present as it is conjugated to conjugated analyte-specific compound 32. The process that would remove conjugated analyte-specific compound 32 if not bound to analyte 16 will not remove conjugated analyte-specific compound 32 in this instance because conjugated analyte-specific compound 32 will be bound to analyte 16. Thus, in this instance, the conjugated analyte-specific compound 32 along with the conjugated enzyme 12 will remain, and when cognate substrate 10 is added, it will react with enzyme 12 to produce a detectable product or compound 18, such as a volatile compound (e.g., ammonia). As shown in FIG. 2a, dye 20, as previously described, is exposed to the detectable compound 18. When exposed to detectable compounds 18, dye 20 undergoes a color change corresponding to the presence of detectable compound 18. The color change of dye 20 can be detected by the human eye 22 or detector 24, as previously described.

As shown in FIG. 2b, the capture analyte-specific compound 30 can be eliminated, thereby forming a single antibody detection assay wherein analyte 16 is placed directly on the surface of a support or well 28.

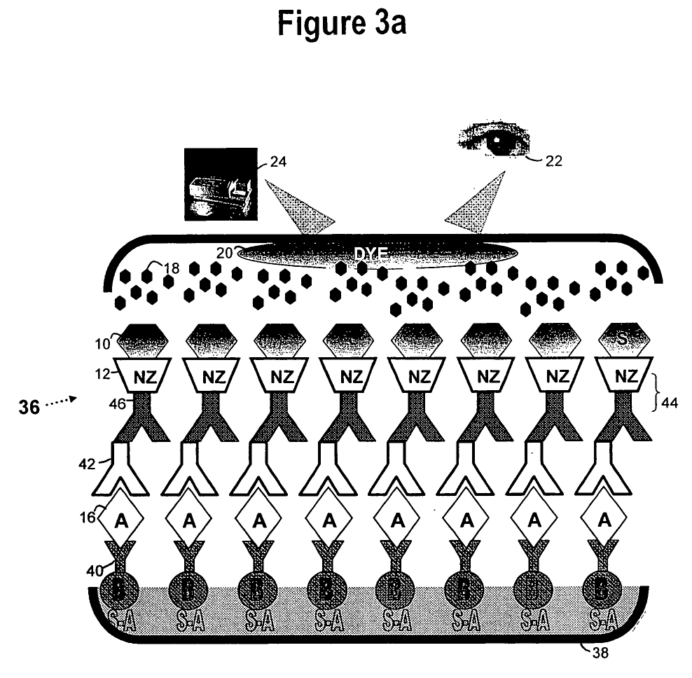

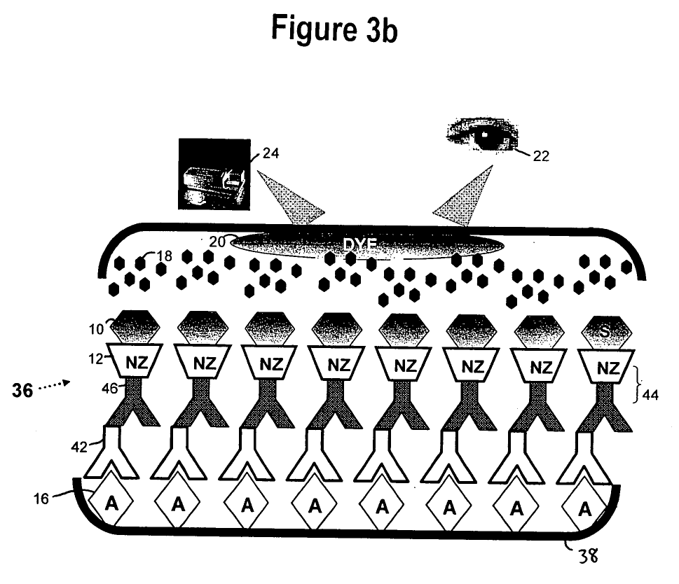

An alternative embodiment of the present invention is shown in FIG. 3a. FIG. 3a illustrates a triple antibody detection assay in accordance with the present invention. An enzyme immunoassay 36 comprises a plate 38, such as a streptavidin coated plate. Enzyme immunoassay 36 also comprises a first antibody 40. First antibody 40 can be a biotinylated antibody. First antibody 40, also called a capture antibody or capture analyte-specific compound, binds to an epitope of analyte 16. A second antibody 42, also called a primary antibody or analyte-specific compound, binds to an epitope of analyte 16 that is distinct from the epitope bound by the capture antibody 40. Conjugate 44 is comprised of a third antibody 46 and enzyme 12. The third antibody 46, which is a non-analyte specific compound, binds to an epitope on the second antibody 42. Enzyme 12 will interact with substrate 10 to produce detectable compound 18.

If analyte 16 is present, binding will occur between the second antibody 42 and analyte 16. This binding will ensure that enzyme 12, which is part of conjugate 44 that is bound to an epitope of second antibody 42, will be present after undergoing a process that would have removed the conjugate 44 had there been no binding between second antibody 42 and analyte 16. Thus, due to the presence of analyte 16, enzyme 12 is present to react with substrate 10 when substrate 10 is added to immunoassay 36, thereby producing detectable compounds 18. As shown in FIG. 3a, dye 20, as previously described, is exposed to detectable compounds 18. When exposed to detectable compounds 18, dye 20 undergoes a color change corresponding to the presence of detectable compounds 18. The color change of dye 20 can be detected by the human eye 22 or detector 24, as previously described.

As shown in FIG. 3b, the first or capture antibody 40 can be eliminated, thereby forming a double antibody detection assay wherein analyte 16 is placed directly on the surface of support or well 38.

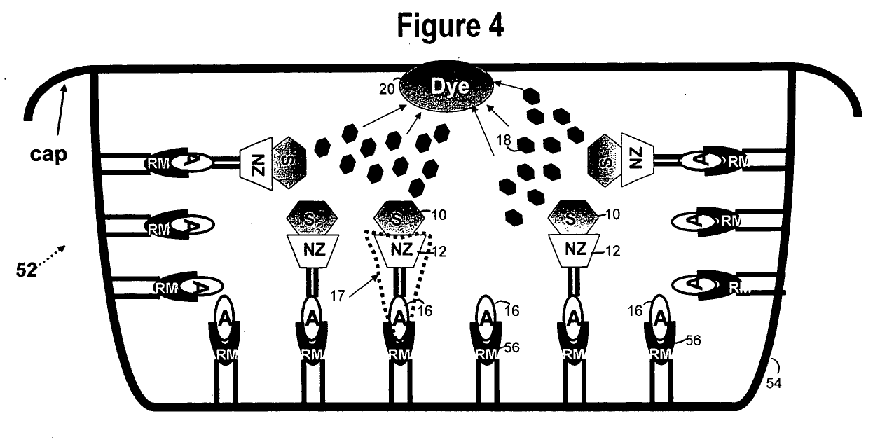

FIG. 4 illustrates an alternative embodiment of the present invention. FIG. 4 illustrates a competitive binding assay test kit 52. Assay test kit 52 can be used to quantitate a given analyte 16. This embodiment uses an analyte 16 conjugated to an enzyme 12 (e.g., urease) as a tracer 17. Assay test kit 52 comprises a tube or well 54. Tube or well 54 is coated with specific receptor molecules 56 that will capture part of an analyte 16 or tracer 17. Molecules of analyte 16 from a patient sample are not labeled with an enzyme 12. The tracer 17 provided in kit 52 is analyte 16 conjugated to enzyme 12. Unlabeled analyte 16 from a patient sample competes with tracer 17 for capture by receptor molecules 56. In other words, analyte 16 molecules originating from a specimen of interest are not labeled with enzyme 12, and therefore they compete for binding with the enzyme conjugated tracer 17 molecules provided as an assay component. Substrate 10, such as urea as an example, is added to the assay tube or well 54, and reacts with the enzyme 12 of the tracer 17 molecules to produce detectable compound 18, such as ammonia. The amount of the detectable compound produced is inversely proportional to the amount of the unlabeled analyte 16 present in the specimen. That is, the more analyte 16 from a patient sample, the less binding by conjugate tracer 17 (i.e., analyte 16 labeled with enzyme 12), and less color change of dye 20. The detectable compound 18 that is produced is detected by the color change of dye 20. The color change of dye 20 can be detected by the human eye 22 or detector 24, as previously described.

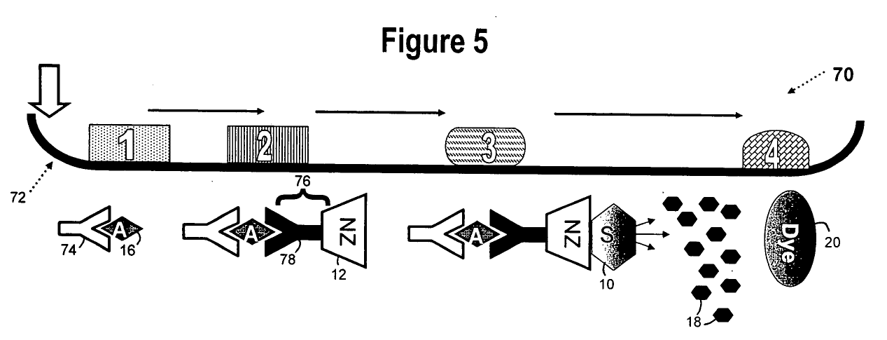

FIG. 5 illustrates a lateral flow assay kit 70 in accordance with the present invention. More specifically, FIG. 5 illustrates the use of an immunoconjugate having an antibody coupled to an enzyme (such as urease) to detect a captured antigen in a lateral flow diagnostic test on a solid support 72. Kit 70 comprises solid support 72, and spots 1, 2, 3 and 4. Lateral flow of a sample from spots 1, 2, 3 through 4 is made possible by any suitable material (e.g., a nitrocellulose membrane). Spot 1 comprises an anti-analyte antibody 74 that binds analyte 16 if present in a sample. Spot 2 comprises an immunoconjugate 76, comprising a second anti-analyte antibody 78 and enzyme 12. Spot 3 comprises a cognate substrate 10. Spot 4 comprises a dye 20. Dye 20 can be located on a strip (not shown) or on the cover (not shown) over substrate 10. If the analyte 16 is present, immunoconjugate or antibody-enzyme complex 76 will bind to analyte 16. When substrate 10, e.g., urea, is added, substrate 10 will react with enzyme 12 to produce detectable compound 18, e.g., ammonia. Detectable compound 18 can then be detected by a color change in a dye 20 due the presence of detectable compound 18. Dye 20 can be located either on the test strip itself or on the inside surface of the cover (not shown) directly above the substrate 10.

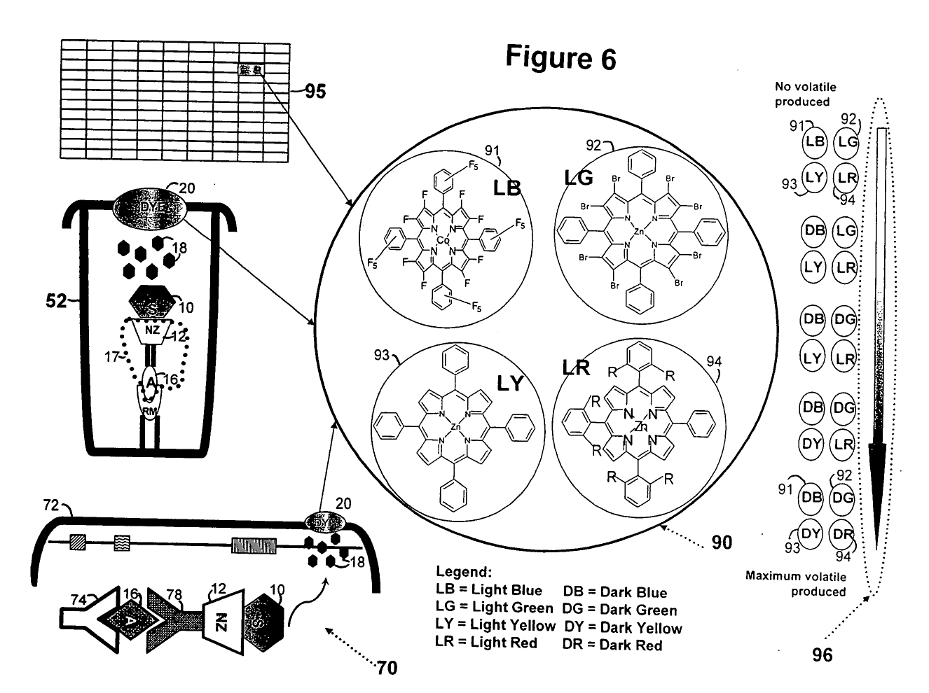

FIG. 6 illustrates an array-based quantification of detectable compound 18 made in accordance with the present invention used to detect analyte 16 in a well of an enzyme immunoassay 26 of FIG. 2 or 36 of FIG. 3, of a competitive binding test kit 52 of FIG. 4, or in a lateral flow assay kit 70 of FIG. 5, all in accordance with the present invention. FIG. 6 shows an array 90. Array 90 comprises a dye 20 as shown and described in FIGS. 1 through 5. In a preferred embodiment, array 90 comprises more than one dye. As shown in FIG. 6, array 90 comprises dyes 91, 92, 93 and 94. Detectable compounds 18 are produced when substrate 10 reacts with the corresponding enzyme 12 as previously described and shown in FIGS. 1 through 5. Array 90 can be situated in the cover over each well of a multi-well enzyme immunoassay 26 or 36, within the cap or cover of a competitive binding test kit 52 or on the strip or cover of a lateral flow assay kit 70. Thus, while FIG. 6 shows competitive binding test kit 52 and lateral flow assay kit 70, an immunoassay 26 or 36, previously described, can also be used. In a more preferred embodiment, array 90 comprises metalloporphyrin dyes, and more preferably metalloporphyrin dyes selected from the group consisting of the dyes identified in formulas I through IV below. Array 90 can be a part of a large array within the cover of a multi-well assay plate, such as a multi-well microtiter plate 95.

In accordance with the present invention, four distinct metalloporphyrin dyes with different binding affinities for a given volatile compound can be used to aid in specific quantitation over a wide dynamic range of the released volatile compound. These metalloporphyrin dyes are only exemplary, and those skilled in the art will recognize that any suitable porphyrin dye or non-porphyrin dye can be used in accordance with the present invention. The four metallporphyrin dyes are as follows:

(I) 2,3,7,8,12,13,17,18,-octafluoro-5,10,15,20-tetrakis(pentafluorophenyl) porphirinatocobalt (II) [Co(F₂₈TPP)]

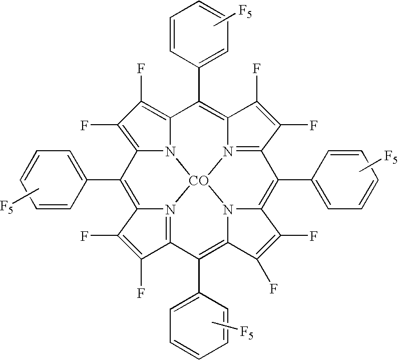

(II) 2,3,7,8,12,13,17,18,-octabromo-5,10,15,20-tetraphenylporphirinatozinc(II) [Zn(Br₈TPP)]

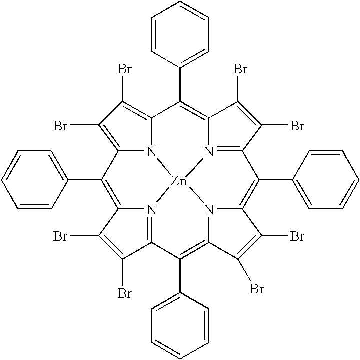

(III) 5,10,15,20-tetraphenylporphirinatozinc(II) [ZnTPP]

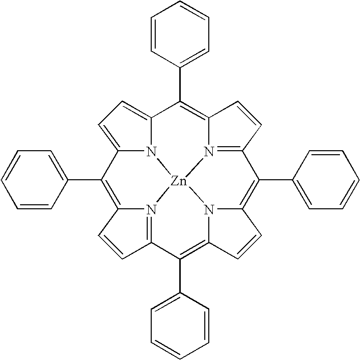

and (IV) 5(phenyl)-10,15,20-trikis(2′,6′-disilyloxyphenyl)porphyrinatozinc(II) [Zn(Si₆PP)]

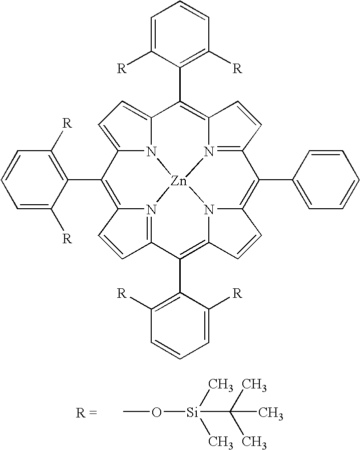

The binding constants (log K) of these metalloporphyrins for pyridine, a detectable volatile compound that is also a Lewis base like ammonia, have been determined as follows:

| Porphyrin | log K | Solvent | Reference |
|---|---|---|---|
| I | 5.9 | Methylene | Smirnov, et al., Inorganic |
| | | Chloride | Chemistry, 37, 4971 (1998) |
| II | 4.8 | Toluene | Bhyrappa, et al., J. Chem. Soc., |
| | | | Dalton Trans., 1901 (1993) |
| III | 3.5 | Toluene | Sen and Suslick, Journal of the |
| | | | American Chemical Society, |
| | | | 122, 11565 (2000), and which |
| | | | can be purchased from Sigma- |
| | | | Aldrich Corp. (of St. Louis, |
| | | | MO) as catalog number 25,217-4 |
| IV | 1.8 | Toluene | Sen and Suslick, Journal of the |
| | | | American Chemical Society, |
| | | | 122, 11565 (2000) |

Based on these binding constants, porphyrin I is the most sensitive to volatile amines while porphyrin IV is the least sensitive to volatile amines. If these four porphyrins are used in concert as depicted in FIG. 6, their color changes will provide a visual key to a wide dynamic range of a given detectable compound 18. Therefore, the present invention provides sensitivity and detection capabilities not currently available. As shown in the scale 96 in FIG. 6, porphyrin I (dye 91) will detect, i.e., undergo a color change, and quantitate a lower amount of detectable compound 18 than porphyrins II, III, and IV (dyes 92, 93, and 94, respectively). Porphyrin II (dye 92) will detect, i.e., undergo a color change, e.g. change from a light color to a dark color, and quantitate a lower amount of detectable compound 18 than porphyrins III, and IV (dyes 93 and 94). Porphyrin III (dye 93) will detect, i.e., undergo a color change, and quantitate a lower amount of detectable compound 18 than porphyrin IV (dye 94). Porphyrin IV (dye 94) will detect, i.e., undergo a color change, and quantitate a higher amount of detectable compound 18 than porphyrins 1, 11, and III (dyes 91, 92, and 93, respectively).

Those of skill in the art will recognize that any suitable metalloporphyrin or metal free porphyrin dye(s) can be used in accordance with the present invention. An example of a particular free-base porphyrin dye suitable for the detection of ammonia in accordance with the present invention is

5,10,15,20-Tetraphenyl-21H,23H-porphine [H₂TPP]

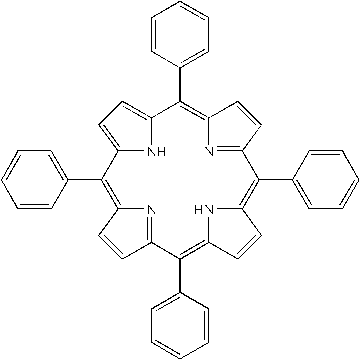

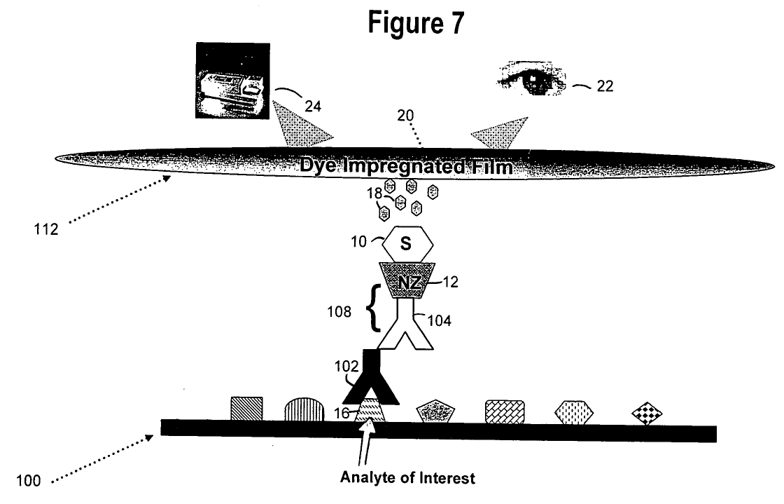

FIG. 7 illustrates the detection of a protein on an Immunoblot membrane 100. By way of example, an analyte 16 of interest can be a protein. A mouse anti-analyte antibody IgG 102 will bind to the analyte 16. A goat anti-mouse IgG antibody 104 will be conjugated to an enzyme 12, forming a goat anti-mouse IgG antibody/enzyme conjugate 108. The antigen binding portion of the goat anti-mouse IgG antibody 104, which is distinct from the portion of the goat anti-mouse IgG antibody 104 conjugated to enzyme 12, will bind to mouse anti-analyte antibody IgG 102. When a cognate substrate 10 is added, it will react with enzyme 12, thereby producing detectable compound 18. A dye impregnated film 112 comprising a responsive dye 20 is exposed to detectable compound 18, thereby changing color of dye 20 directly over analyte 16, i.e. the protein of interest. Film 112, including bands comprised of dyes that have changed color above corresponding proteins of interest, can be scanned and imaged for further use. The color change of dye 20 can be observed by the human eye 22, or a detector 24, as previously described. In accordance with this example of the present invention, film 112 (such as a plasticized film) is impregnated with dye molecules and overlaid above an Immunoblot (Western blot) in order to detect specific proteins of interest. The dye in the film undergoes a visible color change over the protein band of interest if it was present and detected by a specific immunoconjugate. In one embodiment, a substrate, such as urea, can be coformulated with a dye-impregnated film to form an overlay for the detection of enzyme conjugate 108.

In accordance with the present invention, an enzyme can be used to convert a first volatile compound into a second volatile compound, the second volatile compound more readily detectable than the first volatile compound in some circumstances. For example, such an enzymatic conversion serves as an amplification step in producing the second volatile compound, which can be monitored using any suitable detection technique, including but not limited to the methods and devices disclosed in U.S. Pat. Nos. 6,368,558 and 6,495,102, and U.S. Patent Application Publication Nos. US 2003/0129085 A1, US 2003/0143112 A1, and US 2003/0166298 A1, all of which are incorporated herein by reference. By way of example, but not limitation, the enzyme hydrogen-sulfide-S-acetyltransferase (EC 2.3.1.10) converts hydrogen sulfide and acetyl coenzyme A (“CoA”) to thioacetate and CoA. This enzyme and the cofactor acetyl CoA can be immobilized in the device of the present invention to capture a stream of gaseous analytes, or exhaled breath, which in turn causes the conversion of any H₂S present in the sample to thioacetate. The produced thioacetate is then detected by the device of the present invention.

The present invention can be used to detect a wide variety of analytes. The present invention can also be used to detect a wide variety of conditions of a patient. For example, a bodily fluid or tissue can be obtained from a patient and tested by the device and method of the present invention to determine the presence of an analyte indicative of the condition of a patient. The tissue of a patient can be obtained via swab or biopsy.

### EXAMPLE 1

Affinity purified rabbit anti-goat IgG and goat anti-mouse IgG were conjugated to urease using m-maleimidobenzoyl N-hydroxysuccinimate ester as a cross-linking reagent as described (Healy, K., Chandler, H. M., Cox, J. C. and Hurrell, J. G. R. 1983, Clin. Chim. Acta 134:51-58). Aliquots of the IgG-urease conjugates were incubated with 0.05M urea on filter paper that was exposed to a representative sensor array of dye molecules (see e.g., the dye arrays of U.S. Pat. Nos. 6,368,558 and 6,495,102, and U.S. Patent Application Publication Nos. US 2003/0129085 A1, US 2003/0143112 A1, and US 2003/0166298 A1, all of which are incorporated herein by reference). Both the rabbit anti-goat and goat anti-mouse IgG urease conjugates exposed to urea substrate caused substantial color changes to the dyes, thereby confirming the presence of ammonia. These conjugates can be used in the embodiments described in FIGS. 2 through 7. For example, these conjugates can be used to detect human chorionic gonadotropin (“hCG”), a hormone indicative of pregnancy or malignancy.

The specific dyes that showed a substantial color change in accordance with this example were as follows: Thymol Blue, Cresol Red, Phenol Red, Bromothymol Blue, Nitrazine Yellow, Bromocresol Purple, and Bromocresol Green.

The positive response of these dyes was identical to that observed when purified urease enzyme was incubated with 0.05M urea and exposed to these dyes. See FIG. 1. Further, the positive response of these dyes was shown to be specific for ammonia as these dye changes were not detected with urea when a buffer (0.1 M phosphate buffer, pH 6.8) was substituted for conjugate or enzyme.

The enzyme conjugates as described above may be formed by either direct (i.e., covalent) or indirect chemical coupling or tethering of the enzyme to the analyte-specific compound, or non-analyte specific compound, or analyte molecule in the tracer. For example, forming enzyme conjugates by direct chemical coupling may be accomplished using a cross-linking reagent, such as glutaraldehyde, m-maleimidobenzoyl N-hydroxysuccinimide ester (MBS), or via oxidation-reduction using NaIO₄ and NaBH₄ or the like. Forming enzyme conjugates by indirect coupling or tethering of the enzyme to the compound may be accomplished using a tethering linkage having first and second tether compounds where either component of the conjugate is coupled to the first tether compound, and the other component of the conjugate is coupled to the second tether compound. An example of one tethering linkage is a streptavidin-biotin linkage, where either component of the conjugate is coupled to biotin with the other component coupled to streptavidin.

Examples of various combinations of analytes, capture analyte-specific compounds, analyte-specific compounds, and urease conjugates in accordance with the present invention are set forth in the following Table I. Those skilled in the art will recognize that other combinations can be made in accordance with the present invention.

TABLE I

| | Capture Analyte- | Analyte-Specific | |
|---|---|---|---|
| Analyte | Specific Compound | Compound | Urease Conjugate |
| hCG (human chorionic | Mouse monoclonal | Goat polyclonal | Rabbit anti-Goat IgG- |
| gonadotropin) | anti-hCG IgG (with | anti-hCG IgG | Urease Conjugate |
| | or without | | |
| | biotinylation) | | |
| hCG (human chorionic | Goat polyclonal | Mouse monoclonal | Goat anti-Mouse IgG- |
| gonadotropin) | anti-hCG IgG (with | anti-hCG IgG | Urease Conjugate |
| | or without | | |
| | biotinylation) | | |

Many modifications and variations may be made in the techniques and structures described and illustrated herein without departing from the spirit and scope of the present invention. Accordingly, the techniques and structures described and illustrated herein should be understood to be illustrative only and not limiting upon the scope of the present invention.

I/we claim:

1. A device for detecting an analyte comprising an analyte-specific compound that binds to the analyte and produces a detectable compound in combination with a given substrate, said detectable compound producing a response when exposed to at least two dyes, the response being stronger and more distinct than a response of the analyte when exposed to the at least two dyes.

2. The device of claim 1, wherein the analyte-specific compound is chemically coupled to an enzyme.

3. The device of claim 2, further comprising a substrate that chemically reacts with the enzyme to produce the detectable compound.

4. The device of claim 1, wherein the detectable compound that is produced is ammonia.

5. The device of claim 1, wherein the analyte-specific compound is selected from the group consisting of an antibody, protein, biotin, peptide, hapten, specific drug analyte or drug metabolite, carbohydrate moiety, or single stranded or double stranded nucleic acid or from fusion or chimeric molecules comprising structural elements of two or more of these group members such as combinations of nucleic acid and protein or protein and small organic molecules or the like.

6. The device of claim 1, wherein the analyte-specific compound is selected from the group consisting of single stranded nucleic acid and double stranded nucleic acid.

7. The device of claim 6, wherein the single stranded nucleic acid and double stranded nucleic acid is selected from the group consisting of RNA, DNA, base-modified oligonucleotide, chimeric oligonucleotide, amplification reaction product, and cDNA of any length or sequence.

8. The device of claim 2, wherein the enzyme is urease.

9. The device of claim 3, wherein the substrate is urea.

10. The device of claim 8, wherein the substrate is urea.

11. The device of claim 1, further comprising a free enzyme that facilitates the production of the detectable compound.

12. The device of claim 1, further comprising free enzyme and enzyme that is chemically coupled to the analyte-specific compound, wherein each form of enzyme facilitates the production of the detectable compound.

13. The device of claim 1, further comprising at least two dyes that undergo a detectable color change when exposed to the detectable compound.

14. The device of claim 2, wherein the enzyme is placed on a solid support.

15. The device of claim 14, wherein the solid support is selected from the group consisting of a membrane, filter, tube, and well.

16. The device of claim 15, wherein the solid support is made from material comprised of polystyrene or polypropylene.

17. The device of claim 2, wherein the enzyme is in an aqueous or organic solution.

18. The device of claim 13, wherein the at least two dyes are selected from the group consisting of:
2,3,7,8,12,13,17,18,-octafluoro-5,10,15,20-tetrakis (pentafluorophenyl) porphirinatocobalt (II) [Co(F₂₈TPP)];
2,3,7,8,12,13,17,18,-octabromo-5,10,15,20-tetraphenylporphirinatozinc(II) [Zn(Br₈TPP)];
5,10,15,20-tetraphenylporphirinatozinc(II) [ZnTPP];
5(phenyl)-10,15,20-trikis(2′,6′-disilyloxyphenyl)porphyrinatozinc(II) [Zn(Si₆PP)];
5,10,15,20-Tetraphenyl-21H,23H-porphine [H₂TPP];
Thymol Blue; Cresol Red; Phenol Red; Bromothymol Blue; Nitrazine Yellow; Bromocresol Purple; and Bromocresol Green.

19. The device of claim 1, wherein the analyte is selected from the group consisting of an antibody, protein, biotin, peptide, hapten, specific drug analyte or drug metabolite, carbohydrate moiety, or single stranded or double stranded nucleic acid or from fusion or chimeric molecules comprising structural elements of two or more of these group members such as combinations of nucleic acid and protein or protein and small organic molecules or the like.

20. The device of claim 1, wherein the analyte is selected from the group consisting of single stranded nucleic acid and double stranded nucleic acid.

21. The device of claim 20, wherein the single stranded nucleic acid and double stranded nucleic acid is selected from the group consisting of RNA, DNA, base-modified oligonucleotide, chimeric oligonucleotide, amplification reaction product, and cDNA of any length or sequence.

22. A device for detecting an analyte comprising an analyte-specific compound that chemically couples to the analyte and produces a detectable compound, said detectable compound producing a response when exposed to at least two dyes, the response being stronger and more distinct than a response of the analyte when exposed to the at least two dyes.

23. The device of claim 22, wherein the analyte is an enzyme.

24. The device of claim 23, wherein the analyte-specific compound is a substrate.

25. The device of claim 22, wherein the analyte is urease.

26. The device of claim 22, wherein the analyte-specific compound is urea.

27. The device of claim 22, wherein the detectable compound that is produced is ammonia.

28. The device of claim 27, wherein the ammonia is indicative of urease activity in a biological or chemical sample.

29. The device of claim 28, wherein the biological or chemical sample is a bodily fluid or tissue of a patient.

30. The device of claim 29, wherein the bodily fluid of a patient is selected from the group consisting of breath, belch gas, phlegm, urine, feces, blood, saliva, and sweat.

31. The device of claim 29, wherein the tissue is obtained via swab or biopsy.

32. A device for detecting an analyte comprising an analyte-specific compound conjugated to an enzyme, the analyte-specific compound binding to a target site of the analyte, the enzyme producing a detectable compound in combination with a given substrate, the detectable compound producing a detectable response when exposed to at least two dyes, the response being stronger and more distinct than a response of the analyte when exposed to the at least two dyes.

33. The device of claim 32, further comprising a capture analyte-specific compound that is different from the conjugated analyte-specific compound, the capture analyte-specific compound binding to a different target site of the analyte than the conjugated analyte-specific compound.

34. The device of claim 33, wherein the capture analyte-specific compound is bound directly or indirectly to a solid support.

35. A device for detecting an analyte comprising an analyte-specific compound that binds to a target site of the analyte, a conjugate comprising an enzyme and a non-analyte specific compound, the non-analyte specific compound that binds to the analyte-specific compound, the enzyme producing a detectable compound in combination with a given substrate, the detectable compound producing a detectable response when exposed to at least two dyes, the response being stronger and more distinct than a response of the analyte when exposed to the at least two dyes.

36. The device of claim 35, further comprising a capture analyte-specific compound that is different from the analyte-specific compound, the capture analyte-specific compound binding to a different target site of the analyte than the analyte-specific compound.

37. The device of claim 36, wherein the capture analyte-specific compound is bound directly or indirectly to a solid support.

38. A device for detecting an analyte in a sample comprising a receptor molecule for capturing either the free analyte from the sample, or a tracer not from the sample, the tracer comprising an analyte molecule bound to an enzyme, the tracer producing a detectable compound in combination with a given substrate, the detectable compound producing a detectable response when exposed to at least two dyes, the detectable response inversely proportional to the quantity of the analyte in the sample.

39. The device of claim 22, further comprising at least two dyes that undergo a detectable color change when exposed to the detectable compound.

40. The device of claim 39, wherein the at least two dyes are selected from the group consisting of:
2,3,7,8,12,13,17,18,-octafluoro-5,10,15,20-tetrakis (pentafluorophenyl) porphirinatocobalt (II) [Co(F₂₈TPP)];
2,3,7,8,12,13,17,18,-octabromo-5,10,15,20-tetraphenylporphirinatozinc(II) [Zn(Br₈TPP)];
5,10,15,20-tetraphenylporphirinatozinc(II) [ZnTPP];
5(phenyl)-10,15,20-trikis(2′,6′-disilyloxyphenyl)porphyrinatozinc(II) [Zn(Si₆PP)];
5,10,15,20-Tetraphenyl-21H,23H-porphine [H₂TPP];
Thymol Blue; Cresol Red; Phenol Red; Bromothymol Blue; Nitrazine Yellow; Bromocresol Purple; and Bromocresol Green.

41. The device of claim 32, further comprising at least two dyes that undergo a detectable color change when exposed to the detectable compound.

42. The device of claim 41, wherein the at least two dyes are selected from the group consisting of:
2,3,7,8,12,13,17,18,-octafluoro-5,10,15,20-tetrakis (pentafluorophenyl) porphirinatocobalt (II) [Co(F₂₈TPP)];
2,3,7,8,12,13,17,18,-octabromo-5,10,15,20-tetraphenylporphirinatozinc(II) [Zn(Br₈TPP)];
5,10,15,20-tetraphenylporphirinatozinc(II) [ZnTPP];
5(phenyl)-10,15,20-trikis(2′,6′-disilyloxyphenyl)porphyrinatozinc(II) [Zn(Si₆PP)];
5,10,15,20-Tetraphenyl-21H,23H-porphine [H₂TPP];
Thymol Blue; Cresol Red; Phenol Red; Bromothymol Blue; Nitrazine Yellow; Bromocresol Purple; and Bromocresol Green.

43. The device of claim 35, further comprising at least two dyes that undergo a detectable color change when exposed to the detectable compound.

44. The device of claim 43, wherein the at least two dyes are selected from the group consisting of:
2,3,7,8,12,13,17,18,-octafluoro-5,10,15,20-tetrakis (pentafluorophenyl) porphirinatocobalt (II) [Co(F₂₈TPP)];
2,3,7,8,12,13,17,18,-octabromo-5,10,15,20-tetraphenylporphirinatozinc(II) [Zn(Br₈TPP)];
5,10,15,20-tetraphenylporphirinatozinc(II) [ZnTPP];
5(phenyl)-10,15,20-trikis(2′,6′-disilyloxyphenyl)porphyrinatozinc(II) [Zn(Si₆PP)];
5,10,15,20-Tetraphenyl-21H,23H-porphine [H₂TPP];
Thymol Blue; Cresol Red; Phenol Red; Bromothymol Blue; Nitrazine Yellow; Bromocresol Purple; and Bromocresol Green.

45. The device of claim 38, further comprising at least two dyes that undergo a detectable color change when exposed to the detectable compound.

46. The device of claim 45, wherein the at least two dyes are selected from the group consisting of:
2,3,7,8,12,13,17,18,-octafluoro-5,10,15,20-tetrakis (pentafluorophenyl) porphirinatocobalt (II) [Co(F₂₈TPP)];
2,3,7,8,12,13,17,18,-octabromo-5,10,15,20-tetraphenylporphirinatozinc(II) [Zn(Br₈TPP)];
5,10,15,20-tetraphenylporphirinatozinc(II) [ZnTPP];
5(phenyl)-10,15,20-trikis(2′,6′-disilyloxyphenyl)porphyrinatozinc(II) [Zn(Si₆PP)];
5,10,15,20-Tetraphenyl-21H,23H-porphine [H₂TPP];
Thymol Blue; Cresol Red; Phenol Red; Bromothymol Blue; Nitrazine Yellow; Bromocresol Purple; and Bromocresol Green.

47. A method for detecting an analyte comprising the steps of:
a) exposing an analyte-specific compound to an analyte;
b) producing a detectable compound; and
c) exposing the detectable compound to at least two dyes to produce a response, the response being stronger and more distinct than a response of the analyte when exposed to the at least two dyes.

48. A method for detecting an analyte comprising the steps of:
a) exposing an analyte-specific compound to an analyte; and
b) producing a detectable compound, in combination with a given substrate, said detectable compound producing a response when exposed to at least two dyes, the response being stronger and more distinct than a response of the analyte when exposed to the at least two dyes.

49. A method for detecting an analyte comprising the steps of:
a) exposing the analyte to a conjugate, the conjugate comprising an analyte-specific compound conjugated to an enzyme;
b) exposing the conjugate to a given substrate to produce a detectable compound, the detectable compound producing a detectable response when exposed to at least two dyes, the response being stronger and more distinct than a response of the analyte when exposed to the at least two dyes.

50. The method of claim 49, further comprising the step of removing unbound material prior to step b).

51. A method for detecting an analyte comprising the steps of:
a) exposing the analyte to an analyte-specific compound;
b) exposing the analyte-specific compound to a conjugate comprising an enzyme and a non-analyte specific compound, the non-analyte specific compound binding to the analyte-specific compound; and
c) exposing the enzyme to a given substrate to produce a detectable compound, the detectable compound producing a detectable response when exposed to at least two dyes, the response being stronger and more distinct than a response of the analyte when exposed to the at least two dyes.

52. The method of claim 51, further comprising the step of removing unbound material prior to steps b) and c).

53. The device of claim 2, wherein the enzyme that is chemically coupled to the analyte-specific compound is directly coupled to the analyte-specific compound.

54. The device of claim 2, wherein the enzyme that is chemically coupled to the analyte-specific compound is indirectly coupled to the analyte-specific compound.

55. The device of claim 12, wherein the enzyme that is chemically coupled to the analyte-specific compound is directly coupled to the analyte-specific compound.

56. The device of claim 12, wherein the enzyme that is chemically coupled to the analyte-specific compound is indirectly coupled to the analyte-specific compound.

57. The device of claim 23, wherein the enzyme directly couples to the analyte-specific compound.

58. The device of claim 23, wherein the enzyme indirectly couples to the analyte-specific compound.

59. The device of claim 32, wherein the enzyme directly couples to the analyte-specific compound.

60. The device of claim 32, wherein the enzyme indirectly couples to the analyte-specific compound.

61. The device of claim 35, wherein the enzyme directly couples to the non-analyte specific compound.

62. The device of claim 35, wherein the enzyme indirectly couples to the non-analyte specific compound.

63. The device of claim 38, wherein the enzyme directly couples to the analyte molecule of the tracer.

64. The device of claim 38, wherein the enzyme indirectly couples to the analyte molecule of the tracer.

65. The method of claim 49, wherein the enzyme directly couples to the analyte-specific compound.

66. The method of claim 49, wherein the enzyme indirectly couples to the analyte-specific compound.

67. The method claim 51, wherein the enzyme directly couples to the non-analyte specific compound.

68. The device of claim 51, wherein the enzyme indirectly couples to the non-analyte specific compound.

69. A device for detecting an analyte comprising an analyte-specific compound that binds to the analyte and produces a detectable compound in combination with a given substrate, said detectable compound producing a response when exposed to at least one porphyrin dye, the response being stronger and more distinct than a response of the analyte when exposed to the at least one porphyrin dye.

70. The device of claim 69, wherein the analyte-specific compound is chemically coupled to an enzyme.

71. The device of claim 70, further comprising a substrate that chemically reacts with the enzyme to produce the detectable compound.

72. The device of claim 69, wherein the detectable compound that is produced is ammonia.

73. The device of claim 69, wherein the analyte-specific compound is selected from the group consisting of an antibody, protein, biotin, peptide, hapten, specific drug analyte or drug metabolite, carbohydrate moiety, or single stranded or double stranded nucleic acid or from fusion or chimeric molecules comprising structural elements of two or more of these group members such as combinations of nucleic acid and protein or protein and small organic molecules or the like.

74. The device of claim 69, wherein the analyte-specific compound is selected from the group consisting of single stranded nucleic acid and double stranded nucleic acid.

75. The device of claim 74, wherein the single stranded nucleic acid and double stranded nucleic acid is selected from the group consisting of RNA, DNA, base-modified oligonucleotide, chimeric oligonucleotide, amplification reaction product, and cDNA of any length or sequence.

76. The device of claim 70, wherein the enzyme is urease.

77. The device of claim 71, wherein the substrate is urea.

78. The device of claim 76, wherein the substrate is urea.

79. The device of claim 69, further comprising a free enzyme that facilitates the production of the detectable compound.

80. The device of claim 69, further comprising free enzyme and enzyme that is chemically coupled to the analyte-specific compound, wherein each form of enzyme facilitates the production of the detectable compound.

81. The device of claim 69, further comprising at least one porphyrin dye that undergoes a detectable color change when exposed to the detectable compound.

82. The device of claim 70, wherein the enzyme is placed on a solid support.

83. The device of claim 82, wherein the solid support is selected from the group consisting of a membrane, filter, tube, and well.

84. The device of claim 83, wherein the solid support is made from material comprised of polystyrene or polypropylene.

85. The device of claim 70, wherein the enzyme is in an aqueous or organic solution.

86. The device of claim 81, wherein the at least one porphyrin dye is selected from the group consisting of:
2,3,7,8,12,13,17,18,-octafluoro-5,10,15,20-tetrakis (pentafluorophenyl) porphirinatocobalt (II) [Co(F₂₈TPP)];
2,3,7,8,12,13,17,18,-octabromo-5,10,15,20-tetraphenylporphirinatozinc(II) [Zn(Br₈TPP)];
5,10,15,20-tetraphenylporphirinatozinc(II) [ZnTPP];
5(phenyl)-10,15,20-trikis(2′,6′-disilyloxyphenyl)porphyrinatozinc(II) [Zn(Si₆PP)]; and
5,10,15,20-Tetraphenyl-21H,23H-porphine [H₂TPP].

87. The device of claim 69, wherein the analyte is selected from the group consisting of an antibody, protein, biotin, peptide, hapten, specific drug analyte or drug metabolite, carbohydrate moiety, or single stranded or double stranded nucleic acid or from fusion or chimeric molecules comprising structural elements of two or more of these group members such as combinations of nucleic acid and protein or protein and small organic molecules or the like.

88. The device of claim 69, wherein the analyte is selected from the group consisting of single stranded nucleic acid and double stranded nucleic acid.

89. The device of claim 88, wherein the single stranded nucleic acid and double stranded nucleic acid is selected from the group consisting of RNA, DNA, base-modified oligonucleotide, chimeric oligonucleotide, amplification reaction product, and cDNA of any length or sequence.

90. A device for detecting an analyte comprising an analyte-specific compound that chemically couples to the analyte and produces a detectable compound, said detectable compound producing a response when exposed to at least one porphyrin dye, the response being stronger and more distinct than a response of the analyte when exposed to the at least one porphyrin dye.

91. The device of claim 90, wherein the analyte is an enzyme.

92. The device of claim 91, wherein the analyte-specific compound is a substrate.

93. The device of claim 90, wherein the analyte is urease.

94. The device of claim 90, wherein the analyte-specific compound is urea.

95. The device of claim 90, wherein the detectable compound that is produced is ammonia.

96. The device of claim 97, wherein the ammonia is indicative of urease activity in a biological or chemical sample.

97. The device of claim 96, wherein the biological or chemical sample is a bodily fluid or tissue of a patient.

98. The device of claim 97, wherein the bodily fluid of a patient is selected from the group consisting of breath, belch gas, phlegm, urine, feces, blood, saliva, and sweat.

99. The device of claim 97, wherein the tissue is obtained via swab or biopsy.

100. A device for detecting an analyte comprising an analyte-specific compound conjugated to an enzyme, the analyte-specific compound binding to a target site of the analyte, the enzyme producing a detectable compound in combination with a given substrate, the detectable compound producing a detectable response when exposed to at least one porphyrin dye, the response being stronger and more distinct than a response of the analyte when exposed to the at least one porphyrin dye.

101. The device of claim 100, further comprising a capture analyte-specific compound that is different from the conjugated analyte-specific compound, the capture analyte-specific compound binding to a different target site of the analyte than the conjugated analyte-specific compound.

102. The device of claim 101, wherein the capture analyte-specific compound is bound directly or indirectly to a solid support.

103. A device for detecting an analyte comprising an analyte-specific compound that binds to a target site of the analyte, a conjugate comprising an enzyme and a non-analyte specific compound, the non-analyte specific compound that binds to the analyte-specific compound, the enzyme producing a detectable compound in combination with a given substrate, the detectable compound producing a detectable response when exposed to at least one porphyrin dye, the response being stronger and more distinct than a response of the analyte when exposed to the at least one porphyrin dye.

104. The device of claim 103, further comprising a capture analyte-specific compound that is different from the analyte-specific compound, the capture analyte-specific compound binding to a different target site of the analyte than the analyte-specific compound.

105. The device of claim 104, wherein the capture analyte-specific compound is bound directly or indirectly to a solid support.

106. A device for detecting an analyte in a sample comprising a receptor molecule for capturing either the free analyte from the sample, or a tracer not from the sample, the tracer comprising an analyte molecule bound to an enzyme, the tracer producing a detectable compound in combination with a given substrate, the detectable compound producing a detectable response when exposed to at least one porphyrin dye, the detectable response inversely proportional to the quantity of the analyte in the sample.

107. The device of claim 90, further comprising at least one porphyrin dye that undergoes a detectable color change when exposed to the detectable compound.

108. The device of claim 107, wherein the at least one porphyrin dye is selected from the group consisting of:
2,3,7,8,12,13,17,18,-octafluoro-5,10,15,20-tetrakis (pentafluorophenyl) porphirinatocobalt (II) [Co(F₂₈TPP)];
2,3,7,8,12,13,17,18,-octabromo-5,10,15,20-tetraphenylporphirinatozinc(II) [Zn(Br₈TPP)];
5,10,15,20-tetraphenylporphirinatozinc(II) [ZnTPP];
5(phenyl)-10,15,20-trikis(2′,6′-disilyloxyphenyl)porphyrinatozinc(II) [Zn(Si₆PP)]; and
5,10,15,20-Tetraphenyl-21H,23H-porphine [H₂TPP].

109. The device of claim 100, further comprising at least one porphyrin dye that undergoes a detectable color change when exposed to the detectable compound.

110. The device of claim 109, wherein the at least one porphyrin dye is selected from the group consisting of:
2,3,7,8,12,13,17,18,-octafluoro-5,10,15,20-tetrakis (pentafluorophenyl) porphirinatocobalt (II) [Co(F₂₈TPP)];
2,3,7,8,12,13,17,18,-octabromo-5,10,15,20-tetraphenylporphirinatozinc(II) [Zn(Br₈TPP)];
5,10,15,20-tetraphenylporphirinatozinc(II) [ZnTPP];
5(phenyl)-10,15,20-trikis(2′,6′-disilyloxyphenyl)porphyrinatozinc(II) [Zn(Si₆PP)]; and
5,10,15,20-Tetraphenyl-21H,23H-porphine [H₂TPP].

111. The device of claim 103, further comprising at least one porphyrin dye that undergoes a detectable color change when exposed to the detectable compound.

112. The device of claim 111, wherein the at least one porphyrin dye is selected from the group consisting of:
2,3,7,8,12,13,17,18,-octafluoro-5,10,15,20-tetrakis (pentafluorophenyl) porphirinatocobalt (II) [Co(F₂₈TPP)];
2,3,7,8,12,13,17,18,-octabromo-5,10,15,20-tetraphenylporphirinatozinc(II) [Zn(Br₈TPP)];
5,10,15,20-tetraphenylporphirinatozinc(II) [ZnTPP];
5(phenyl)-10,15,20-trikis(2′,6′-disilyloxyphenyl)porphyrinatozinc(II) [Zn(Si₆PP)]; and
5,10,15,20-Tetraphenyl-21H,23H-porphine [H₂TPP].

113. The device of claim 106, further comprising at least one porphyrin dye that undergoes a detectable color change when exposed to the detectable compound.

114. The device of claim 113, wherein the at least one dye is selected from the group consisting of:
2,3,7,8,12,13,17,18,-octafluoro-5,10,15,20-tetrakis (pentafluorophenyl) porphirinatocobalt (II) [Co(F₂₈TPP)];
2,3,7,8,12,13,17,18,-octabromo-5,10,15,20-tetraphenylporphirinatozinc(II) [Zn(Br₈TPP)];
5,10,15,20-tetraphenylporphirinatozinc(II) [ZnTPP];
5(phenyl)-10,15,20-trikis(2′,6′-disilyloxyphenyl)porphyrinatozinc(II) [Zn(Si₆PP)]; and
5,10,15,20-Tetraphenyl-21H,23H-porphine [H₂TPP].

115. A method for detecting an analyte comprising the steps of:
a) exposing an analyte-specific compound to an analyte;
b) producing a detectable compound; and
c) exposing the detectable compound to at least one porphyrin dye to produce a response, the response being stronger and more distinct than a response of the analyte when exposed to the at least one porphyrin dye.

116. A method for detecting an analyte comprising the steps of:
a) exposing an analyte-specific compound to an analyte; and
b) producing a detectable compound, in combination with a given substrate, said detectable compound producing a response when exposed to at least one porphyrin dye, the response being stronger and more distinct than a response of the analyte when exposed to the at least one porphyrin dye.

117. A method for detecting an analyte comprising the steps of:
a) exposing the analyte to a conjugate, the conjugate comprising an analyte-specific compound conjugated to an enzyme;
b) exposing the conjugate to a given substrate to produce a detectable compound, the detectable compound producing a detectable response when exposed to at least one porphyrin dye, the response being stronger and more distinct than a response of the analyte when exposed to the at least one porphyrin dye.

118. The method of claim 117, further comprising the step of removing unbound material prior to step b).

119. A method for detecting an analyte comprising the steps of:
a) exposing the analyte to an analyte-specific compound;
b) exposing the analyte-specific compound to a conjugate comprising an enzyme and a non-analyte specific compound, the non-analyte specific compound binding to the analyte-specific compound; and
c) exposing the enzyme to a given substrate to produce a detectable compound, the detectable compound producing a detectable response when exposed to at least one porphyrin dye, the response being stronger and more distinct than a response of the analyte when exposed to the at least one porphyrin dye.

120. The method of claim 119, further comprising the step of removing unbound material prior to steps b) and c).

121. The device of claim 70, wherein the enzyme that is chemically coupled to the analyte-specific compound is directly coupled to the analyte-specific compound.

122. The device of claim 70, wherein the enzyme that is chemically coupled to the analyte-specific compound is indirectly coupled to the analyte-specific compound.

123. The device of claim 80, wherein the enzyme that is chemically coupled to the analyte-specific compound is directly coupled to the analyte-specific compound.

124. The device of claim 80, wherein the enzyme that is chemically coupled to the analyte-specific compound is indirectly coupled to the analyte-specific compound.

125. The device of claim 91, wherein the enzyme directly couples to the analyte-specific compound.

126. The device of claim 91, wherein the enzyme indirectly couples to the analyte-specific compound.

127. The device of claim 100, wherein the enzyme directly couples to the analyte-specific compound.

128. The device of claim 100, wherein the enzyme indirectly couples to the analyte-specific compound.

129. The device of claim 103, wherein the enzyme directly couples to the non-analyte specific compound.

130. The device of claim 103, wherein the enzyme indirectly couples to the non-analyte specific compound.

131. The device of claim 106, wherein the enzyme directly couples to the analyte molecule of the tracer.

132. The device of claim 106, wherein the enzyme indirectly couples to the analyte molecule of the tracer.

133. The method of claim 117, wherein the enzyme directly couples to the analyte-specific compound.

134. The method of claim 117, wherein the enzyme indirectly couples to the analyte-specific compound.

135. The method claim 119, wherein the enzyme directly couples to the non-analyte specific compound.

136. The device of claim 119, wherein the enzyme indirectly couples to the non-analyte specific compound.
